(19) United States
(12) Patent Application Publication
(10) Pub. No.: US 2012/0095544 A1
(43) Pub. Date: Apr. 19, 2012

(54) STENT MOVEMENT PREVENTING DEVICE

(76) Inventor: Andrew Jeffrey, Tubingen (DE)

(21) Appl. No.: 13/055,292

(22) PCT Filed: May 18, 2009
(86) PCT No.: PCT/EP2009/003525

§ 371 Date: Apr. 25, 2011

(30) Foreign Application Priority Data
Jul. 23, 2008 (EP) 08013292.1

(51) Int. Cl. A61F 2/84; A61F 2/82

(52) U.S. Cl. 623/1.12; 623/1.16

(57) ABSTRACT

A device for preventing stent movement during delivery including a securement connector arranged to engage a catheter, at least one flexible connecting member, the flexible connecting member having a first end portion and a second end portion, the first end portion coupled to the securement connector, and a locking member located at the flexible connecting member second end portion arranged to engage a portion of a stent including struts defining gaps therebetween wherein the locking member takes the form of a clasp section by snapping into one gap of the gaps between the struts.

### CROSS-REFERENCE TO RELATED APPLICATIONS

This application is a U.S. Nationalization of PCT Application Number PCT/EP2009/003525 filed May 18, 2009 which claims the benefit of European Patent Application No. 08013292.1 filed Jul. 23, 2008, the entireties of which are incorporated herein by reference.

### BACKGROUND

1. Field of Invention

The present invention relates to a device for preventing stent movement during delivery into a lumen of a body vessel and a stent delivery system.

2. Relevant Technology

A mechanism to improve stent securement is known from US 2004/0236406 A1. This device comprises a securement connector arranged to engage a catheter, at least a flexible connecting member coupled to the securement connector and a locking member arranged to engage a portion of a stent. For the engagement of the locking member with the stent, however, an associated engageable portion formed on at least one end of the stent has to be provided. Thus the device is only usable with a modified type of stent.

### BRIEF SUMMARY

It is, therefore, an object of the present invention to provide a device for preventing stent movement that can be used without additional engageable portions on the stent to be provided, and, therefore, simplifies the overall connecting structure to prevent a stent movement.

The solution of this object may be achieved by at least one embodiment of the present invention. A device for preventing stent movement according to one embodiment of the present invention includes a securement connector arranged to engage a catheter and at least one flexible connecting member. The flexible connecting member includes a first portion and a second portion. The first portion is coupled to the securement connector. Further, the device for preventing stent movement includes a locking member located at the flexible connecting member second end portion. This locking member is arranged to engage a portion of a stent having struts defining gaps therebetween. The locking member takes the form of a clasp section by snapping into one of the gaps between the struts.

In a further embodiment of the invention, a stent delivery system is disclosed that includes a catheter, an expandable balloon, and a radially expandable stent having at least one engageable portion which is characterized by a device for preventing stent movement according to at least one embodiment of the present invention, which includes a securement connector arranged to engage a catheter and at least one flexible connecting member. The flexible connecting member has a first portion and a second portion. The first portion is coupled to the securement connector. Further, the device for preventing stent movement includes a locking member located at the flexible connecting member second end portion. This locking member is arranged to engage a portion of a stent having struts that define gaps therebetween. The locking member takes the form of a clasp section by snapping into one of the gaps between the struts. So, as a specific advantage, a standard type stent without any modifications can be secured by simply using the gaps of the stent structure as the technical feature cooperating with at least one embodiment of the clasp section of the locking member.

A method of assembling a device for preventing movement of a stent is described. The method includes mounting the stent in a pre-crimped state over a balloon working length of a balloon; positioning the stent over clasp sections of locking members of the device for preventing stent movement during delivery and for ensuring that the clasp sections are aligned between the struts of the stent. The method includes inserting the entire assembly of the stent and the preventing device into a crimp head of a crimping device crimped in the entire assembly to its nominal diameter.

### BRIEF DESCRIPTION OF THE DRAWINGS

Further features and advantages of the present invention will become apparent from the following description of preferred embodiments with reference to the appended drawings, wherein

FIG. 1 shows an enlarged perspective view of the device for preventing stent movement according to the present invention,

FIG. 2 shows an enlarged cross sectional view of a distal part of a catheter including the device for preventing stent movement according to the present invention in an inflated state of the balloon,

FIG. 3 shows an enlarged cross sectional view of a stent delivery system according to the present invention in a deflated state of the balloon, and

FIG. 4 shows an enlarged perspective view of a further embodiment of the device for preventing stent movement according to the present invention.

### DETAILED DESCRIPTION

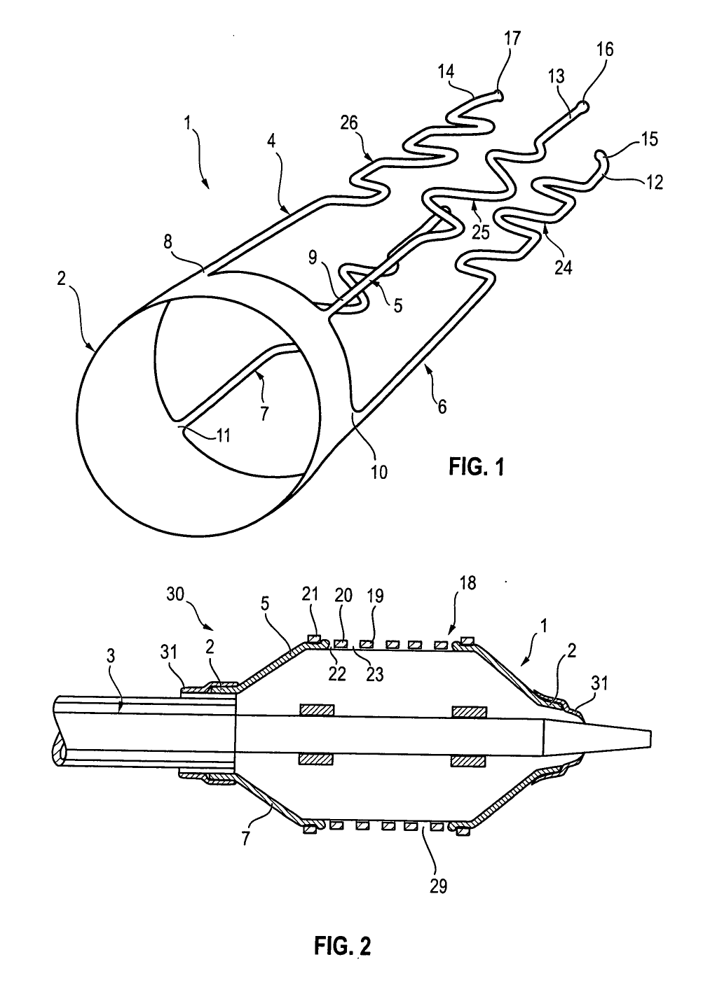

In the perspective view of FIG. 1 the device 1 for preventing stent movement is depicted. The device 1 includes on its proximal end a securement connector 2 in the form of a ring to be connected with a catheter 3. A plurality of flexible connecting members 4, 5, 6, 7 are connected on respective first end portions 8, 9, 10, 11 with the securement connector 2 and extend in a distal direction via serpentine bands 24, 25, 26 towards respective second end portions 12, 13, 14, of the device 1. The second end portions 12, 13, 14, are formed with locking members 15, 16, 17 for the engagement with a portion of a stent 18 not shown in FIG. 1.

FIG. 2 shows an enlarged cross sectional view of a distal part of a catheter including the device for preventing stent movement according to an embodiment of the present invention in an inflated state of a balloon 29. As can be seen from FIG. 2, both a first securement connector 2 at the proximal end of the balloon 29 and a second securement connector 2 at the distal end of the balloon are employed to fix the stent 18 each using connecting members 4-7. Due to this cross sectional view only the connecting members 5, 7 are visible whilst the other connecting members 4, 8 are not visible. The securement connector 2 of each device 1 is connected with the catheter 3 via a shrink tube 31, respectively. The connecting members 4-7 are flexible and expand with the balloon 29 via their serpentine bands 24-26 as illustrated in the inflated state of the balloon 29 in FIG. 2. The locking members 15, 16, 17 formed at the second end portions 8-11 of the connecting members 4-7, respectively, comprise the form of a clasp section by snapping, as shown in FIG. 2, e. g. into one gap 22 of gaps 22, 23 between two adjacent struts 20, 21 of the struts 19-21 of the stent 18 being identified by reference signs representative for all struts and gaps of the stent. Thus, without the need of modifications of the stent structure the stent 18 is fixed from the proximal end as well as the distal end via the device 1, respectively and reliably prevented from moving.

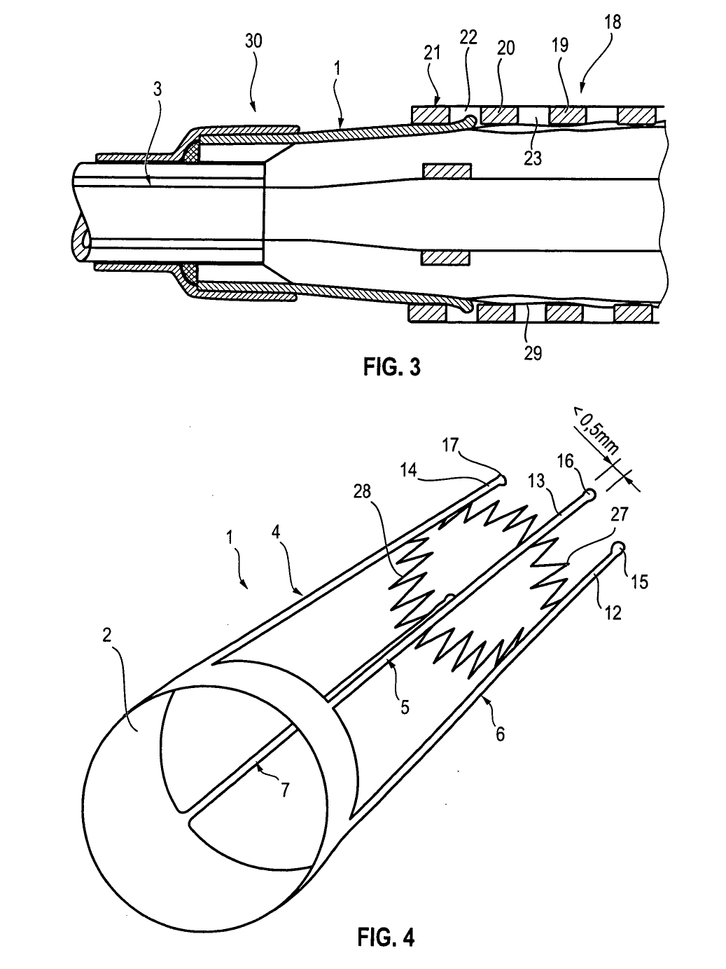

FIG. 3 shows an enlarged cross sectional view of stent delivery system 30 according to an embodiment of the present invention including the catheter 3, the expandable balloon 29 and the radially expandable stent 18 in a deflated state of the balloon 29. When the balloon 29 is deflated the flexible connecting members 4-7 which are formed from a shape memory material (or other flexible material like nitinol or an elastic polymer) bend back and retain their generally linear shape, as shown in FIG. 1. The locking members 15-17 on their second end portions 12-14 remain engaged within respective gaps 22, 23 between the struts 19-21 of the stent 18. In FIG. 3, the same connecting configuration as in FIG. 1 is exemplarily depicted with the two connecting members 5, 7 snapping into the gap 22 between the struts 20, 21 of the stent 18. However, upon retraction of catheter 3 the locking members 15-17 easily slide out of the respective gaps 22 in order to release stent 18.

FIG. 4 shows an enlarged perspective view of a further embodiment of the device for preventing stent movement according to an embodiment the present invention. In this embodiment, highly flexible prongs 27, 28 are provided to connect the connecting members 4-7 in a region adjacent to their second end portion 12-14 for a further improved fixation of a non-modified stent 18.

In addition to the written disclosure above, reference is herewith made explicitly to the disclosure of the invention in FIGS. 1 to 4.

1. A device for limiting movement of a stent during delivery, the stent including a portion having struts, the struts defining gaps therebetween, the device comprising:
a securement connector arranged to engage a catheter;
at least one flexible connecting member, said at least one flexible connecting member having a first end portion and a second end portion, said first end portion coupled to said securement connector, and
a locking member located at said second end portion of said flexible connecting member and arranged to engage a portion of the struts of the stent;
wherein said locking member takes the form of a clasp section by snapping into one of the gaps between the struts.

2. The device of claim 1, further comprising a plurality of flexible connecting members.

3. The device of claim 2, wherein at least one of said flexible connecting members is adapted to expand with the stent.

4. The device of claim 3, wherein each of said flexible connecting members comprises a serpentine band being disposed between said first end portion and said locking member.

5. The device of claim 4, wherein said flexible connecting member is balloon expandable.

6. The device of claim 5, wherein said flexible connecting member is made from a shape memory material.

7. The device of claim 6, wherein said shape memory material has an Af temperature lower than normal human body temperature.

8. The device of claim 7, further comprising a shrink tube that connects said securement connector with the stent.

9. The device of claim 8, wherein prongs connect said flexible connecting members in a region adjacent to said second end portions of said flexible connecting members.

10. A stent delivery system comprising:
a catheter;
an expandable balloon,
a radially expandable stent having at least one engageable portion; and
a device for limiting movement of a stent during delivery comprising:
a securement connector arranged to engage a catheter;
at least one flexible connecting member, said at least one flexible connecting member having a first end portion and a second end portion, said first end portion coupled to said securement connector, and
a locking member located at said second end portion of said flexible connecting member and arranged to engage a portion of the struts of the stent;
wherein said locking member takes the form of a clasp section by snapping into one of the gaps between the struts.

11. The stent delivery system of claim 10, wherein said device and said engagable portion of said stent are adapted to remain engaged until said balloon is deflated.

12. A method of securing a stent to limit movement of a stent during stent delivery, the stent including a stent body having struts, the method comprising:
mounting the stent in a pre-crimped state over a balloon working length of a balloon;
positioning the stent over a preventing device having a plurality of clasp sections of a plurality of locking members of the device for limiting stent movement during delivery and for ensuring that the clasp sections are aligned between the struts of the stent;
inserting the stent and the preventing device into a crimp head of a crimping device in which the entire assembly is crimped to its nominal diameter.
